(19) United States
(12) Patent Application Publication
(10) Pub. No.: US 2022/0049321 A1
(43) Pub. Date: Feb. 17, 2022

(54) METHOD, SYSTEM AND APPARATUS FOR BLOOD PROCESSING UNIT

(71) Applicant: Tangen Biosciences, Inc., Branford, CT (US)

(72) Inventors: John F. Davidson, Guilford, CT (US); Zheng Xue, Madison, CT (US); David Richards, New Haven, CT (US)

(73) Assignee: Tangen Biosciences, Inc., Branford, CT (US)

(21) Appl. No.: 17/513,812

(22) Filed: Oct. 28, 2021

Related U.S. Application Data
(63) Continuation-in-part of application No. 17/400,136, filed on Aug. 12, 2021.
Application No. 17/400,136 is a continuation-in-part of application No. 15/522,039, filed on Apr. 26, 2017.
(60) Provisional application No. 63/117,446, filed on Nov. 23, 2020.
(60) Provisional application No. 63/117,434, filed on Nov. 23, 2020.
(60) Provisional application No. 63/117,446, filed on Nov. 23, 2020.
(60) Provisional application No. 63/117,442, filed on Nov. 23, 2020.
(60) Provisional application No. 63/117,434, filed on Nov. 23, 2020.
(60) Provisional application No. 62/074,325, filed on Nov. 3, 2014.

(51) Int. Cl. C12Q 1/70; G01N 1/40; C12Q 1/6806

(52) U.S. Cl. CPC C12Q 1/70; G01N 1/4044; G01N 1/4077; G01N 2001/4016; G01N 1/4005; G01N 2001/4088; C12Q 1/6806

(57) ABSTRACT

The disclosed embodiments may be used, among others, to extract particles from blood. The particles may include pathogens, viruses, bacteria and other microorganisms present in mammalian blood. An embodiment of the disclosure relates to a system to detect one or more blood-borne pathogens. The exemplary system includes: a transfer assembly having a tube and a hallow needle, the hallow needle centrally located within the transfer assembly tube and configured to communicate a sample material therethrough; a lysing syringe to couple to the transfer assembly, the lysing syringe comprising one or more lysing reagent and a plunger activatable to receive the sample material through the transfer assembly; and a large volume concentrator (LVC) to sealingly couple to the lysing syringe and to separate at least one pathogen from the sample material, the LVC further comprising: a filter support, a membrane, a retainer and a threaded portion.

The disclosure claims priority to the U.S. Provisional Application No. 61/117,434, filed Nov. 23, 2020 (entitled “Method, System and Apparatus for Bloor Processing Unit”), to U.S. Provisional Application No. 63/117,442, filed Nov. 23, 2020 (entitled “Method, System and Apparatus for Respiratory Testing”) to U.S. Provisional Application No. 63/117,446 filed Nov. 23, 2020 (entitled “Method, System and Apparatus for Detection”), to International Application No. PCT/US21/45630, filed Aug. 12, 2021 (entitled “Method, System and Apparatus for Detection”), and to U.S. application Ser. No. 17/400,136, filed. Aug. 12, 2021 (entitled “Method; System and Apparatus for Detection”); the disclosure is also a Continuation-In-Part (“OP”) of and claims priority to application Ser. No. 15/522,039, filed Apr. 26, 2017 (entitled “Apparatus and Method for Cell, Spore, or Virus Capture and Disruption”) and International Application No/PCT/US2015/058612, filed Nov. 2, 2015 (entitled “Apparatus and Method for Cell, Spore, or Virus Capture and Disruption”) which both claim priority to U.S. Provisional Application No. 62/074,325, filed Nov. 3, 2014. The disclosures of all of the preceding applications are incorporated herein in their entireties.

### FIELD

The disclosure relates generally to method, system and apparatus pertaining to a blood processing unit. The disclosed embodiments may be used, among others, to extract particles from blood. The particles may include pathogens, viruses, bacteria and other microorganisms present in mammalian blood.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Nucleic acid analysis methods based on the complementarity of nucleic acid nucleotide sequences can analyze genetic traits directly. Thus, these methods are a very powerful means for identification of genetic diseases, cancer, microorganisms etc. Nucleic acid amplification technologies (NAAT) allow detection and quantification of a nucleic acid in a sample with high sensitivity and specificity. NAAT techniques may be used to determine the presence of a particular template nucleic acid in a sample, as indicated by the presence of an amplification product (i.e., amplicon) following the implementation of a particular NAAT. Conversely, the absence of any amplification product indicates the absence of template nucleic acid in the sample. Such techniques are of great importance in diagnostic applications, for example, for determining whether a pathogen is present in a sample. Thus, NAAT techniques are useful for detection and quantification of specific nucleic acids for diagnosis of infectious and genetic diseases.

Identification of pathogens via direct detection of specific and unique DNA or RNA sequences has been exploited for clinical diagnostic purposes for some time. Molecular detection technologies typically have high analytical sensitivity and specificity compared to antigen and antibody-based methods. Detection of specific genomic DNA or RNA is achieved via amplification of small unique regions of the genome via NAATs such as polymerase chain reaction (PCR, RT-PCR) as well as isothermal methods including loop mediated isothermal amplification (LAMP, RT-LAMP), nucleic acid sequence-based amplification (NASBA), nicking enzyme amplification reaction (NEAR) and rolling circle amplification (RCA), for example. In the case of PCR based amplification, the need for rapid temperature thermocycling and purified sample restricts the use of the technology to a laboratory environment and limits the minimum cost, size and portability.

LAMP, unlike PCR, does not require rapid temperature cycling and so the power demands of the instrument are much lower. This enables a low-cost alternative to the traditional lab-based PCR thermocycler. In addition, LAMP has a short time to positivity—as fast as 5 minutes for strongly positive samples and the degree of sample purity required is much lower while still having analytical sensitivity comparable or superior to PCR. In order to detect RNA, a LAMP based system requires an enzyme or enzymes that can reverse transcribe the RNA template before LAMP amplification and detection. The RT-LAMP assay can therefore be either 2-step, with the first step being a dedicated reverse transcriptase enzyme copying the RNA template into cDNA followed by the geometric LAMP amplification of the target, or preferably a single enzyme RT-LAMP process such as the LavaLAMP™ enzyme from Lucigen Inc., Middleton, Wis.

Upper respiratory tract infections are usually detected by taking swabs from the nasal, nasopharyngeal or throat and eluting the virus from them. The preparation of the RNA for detection by PCR requires further purification to remove contaminants that are less inhibitory to LAMP reactions. This enables a rapid and easy sample preparation for LAMP based assays—a requirement for simple point of care use. In the case of swab, directly eluting the virus into a suitable assay buffer and directly putting that sample into the molecular test system with a simple transfer step is enabling for point of care operation.

For a point of care device, speed and simplicity of use are requirements. No precise measuring during operation or requirements for environmental temperature and humidity are preferred, and the reagents should ideally not require freezing or refrigerated storage. Tight temperature control, automatic fluidic staging and real time monitoring of the LAMP reaction with software to analyze the reaction and report the results to the user is preferred. Bringing the speed and sensitivity of LAMP together with an automated system that is designed to allow for operation outside of a laboratory with simple to use operating steps and room temperature reagents, is a powerful point of care combination. A single enzyme RT-LAMP system reduces assay time as reverse transcription and LAMP amplification occur simultaneously and allows for detection of RNA based pathogens including the majority of respiratory viruses such as influenza A and B, coronaviruses including SARS-CoV-2, and Respiratory Syncytial Virus (RSV).

A system that was able to look for a panel of multiple potential virus pathogens from a single sample would enable definitive diagnosis of the common early upper respiratory symptoms; sore throat, cough, mild fever and running nose to distinguish serious infections such as Sars-CoV-2 or influenza from mild disease caused by rhinovirus or adenovirus, for example. The Tangen GeneSpark™ (Branford, Conn.) instrument was designed with all these features in mind—rapid highly accurate LAMP amplification detection with a low-cost disposable assay disk that affords a panel of up to 32 different pathogen targets from a single patient sample and portability, connectivity and ease of use to allow for point of care results. The SARS-Cov-2 pandemic has underscored the pressing need for rapid accurate testing outside of the laboratory setting at the point of care, with the information getting immediately the patient so they can manage their exposure to others, as well delivering the result to public health databases, so that the pandemic can be tracked, traced and controlled.

Identification of pathogens by testing mammalian blood has been extensively studied. The conventional methods use various detection methods for sampling and analyzing the blood stream. The conventional methods generally lack simplicity and reproducibility.

### BRIEF DESCRIPTION OF THE DRAWINGS

Certain of the above-disclosed embodiments are illustrated in the following schematic representation. It should be noted that these representations are exemplary and non-limiting. Any modification of these exemplary embodiments may be made without departing from the disclosed principles. The illustrated exemplary figures, in which similar elements may be identified similarly, include:

FIG. 1 shows components of an exemplary system or kit for Blood Processing System (BPS) and for sample preparation;

FIG. 2A illustrates an exemplary LVC according to one embodiment of the disclosure;

FIG. 2B is an exploded illustration of the LVC of FIG. 2A;

FIG. 3 schematically illustrates the sample extraction steps according to one embodiment of the disclosure;

FIG. 4 illustrates an exemplary embodiment for implementing the lysing step according to one embodiment of the disclosure;

FIG. 5 illustrates the exemplary steps for separating the pathogenic components of interest from the sample according to one embodiment of the disclosure; and

FIG. 6 illustrates the syringe removal and membrane filter wash process according to one embodiment of the disclosure.

### DETAILED DESCRIPTION

The disclosed embodiments generally relate to system, method and apparatus for extracting particles from mammalian blood. Once extracted, the particles may be further processed to identify the presence (or absence) of a disease. The further processing of the particles may include, lysing a cell associated with the particle to extract the cells nucleic acid and sequencing the cell's nucleic acid to determine its identity. As referenced herein, a particle may include, but is not limited to, bacteria, virus and viral microorganism, spores, or fungi present in the sample (collectively, pathogens).

The disclosure also includes kits for detecting or quantifying a target nucleic acid in a blood sample. An exemplary kit includes (i) a blood processing unit to extract particles from mammalian blood; (ii) sample prep section for separating nucleic acid associated with the extracted particles; (iii) a solid phase disc for identifying nucleic acids having one or more amplification primer sets and one or more second primer sets; and (iv) instructions for use of the disk for a method of detecting a microorganism in a nucleic acid sample from a subject on an apparatus, instrument, or system described herein.

Various aspects of the invention will now be described with reference to the following section which will be understood to be provided by way of illustration only and not to constitute a limitation on the scope of the invention.

In some embodiments, an apparatus and methods for rapid isolation, concentration, and purification of microbes/pathogens of interest from a raw biological sample (i.e., biological specimen) such as blood is described. Samples may be processed directly from biological or clinical sample collection vessels, such as vacutainers, by coupling with the sample processing apparatus in such a manner that minimizes or eliminates user exposure and potential contamination issues.

In an exemplary embodiment, the apparatus comprises a staged syringe or piston arrangement configured to withdraw a desired quantity of biological sample from a sample collection vessel. The sample is then mixed with selected processing reagents to prepare the sample for isolation of viruses, microbes, solids or pathogens (collectively, pathogens) contained therein.

Sample processing may include liquefying or homogenizing non-pathogenic components of the biological specimen and performing various fluidic transfer operations induced by operation of the syringe or piston. The resulting sample (i.e., processed sample) constituents may be redirected to flow across a capture filter or membrane of appropriate size or composition to capture specific microbes/pathogens or other biological sample constituents.

Additional operations may be performed including washing and drying of the filter or membrane by action of the syringe or piston. In various embodiments, sample backflow and cross-contamination within the device is avoided using one-way valves that direct sample fluids along desired paths while preventing leakage, backflow, and/or undesired sample movement.

An exemplary device may include a capture filter for retaining microbes/pathogens (i.e., pathogenic components) of interest allowing them to be readily separated from sample eluent or remaining fraction of the processed sample/waste. The capture filter may be housed in a sealable container and can further be configured to be received directly by other sample processing/analytical instruments for performing downstream operations such as lysis, elution, detection and identification of the captured microbes/pathogens retained on the filter membrane.

The collector may comprise various features to facilitate automated or semi-automated sample processing and include additional reagents contained in at least one reservoir integrated into the collector to preserve or further process the isolated microbes/pathogens captured or contained by the filter/membrane. In various embodiments, the collector may contain constituents capable of chemically disinfecting the isolated microbes/pathogens or render the sample non-infectious while preserving the integrity of biological constituents associated with the microbe/pathogen such as nucleic acids and/or proteins that may be desirably isolated for subsequent downstream processing and analysis. The collector and associated instrument components may desirably maintain the sample in an isolated environment avoiding sample contamination and/or user exposure to the sample contents.

In various embodiments, the disclosure describes an apparatus that permits rapid and semi-automated isolation and extraction of microorganisms such as bacteria, virus, spores, and fungi or constituent biomolecules associated with the microorganisms, such as nucleic acids and/or proteins from a biological sample without extensive hands-on processing or lab equipment. The apparatus has the additional benefit of concentrating the microbes, pathogens, or associated biomolecules/biomaterial of interest. For example, bacteria, virus, spores, or fungi present in the sample (or nucleic acids and/or proteins associated therewith) may be conveniently isolated from the original sample material and concentrated on the filter or membrane. Concentration in this manner increases the efficiency of the downstream assays and analysis improving detection sensitivity by providing lower limits of detection relative to the input sample.

The sample preparation apparatus of the instant disclosure may further be adapted for use with analytical devices and instruments capable of processing and identifying the microorganisms and/or associated biomolecules present within the biological sample. In various embodiments, the sample collector and various other components of the system can be fabricated from disposable materials such as molded plastic that are compatible with downstream sample processing methods and economical to produce. Such components may be desirably sealed and delivered in a sterile package (e.g., a kit or s a system) for single use to thereby avoid potential contamination of the sample contents or exposure of the user while handling. In various embodiments, the reagents of the sample collector provide for disinfection of the sample constituents such that may be disposed of without risk or remaining infectious or hazardous. The sample collector provides simplified workflows and may not require specialized training or procedures for handling and disposal.

In various embodiments, the automated and semi-automated processing capabilities of the system simplify sample preparation and processing protocols. A practical benefit may be realized in an overall reduction in the number of required user operations, interactions, or potential sample exposures as compared to conventional sample processing systems. This results in lower user training requirements and fewer user-induced failure points. In still other embodiments, the system advantageously provides effective isolation and/or decontamination of a sample improving overall user safety while at the same time preserving sample integrity, for example by reducing undesirable sample degradation.

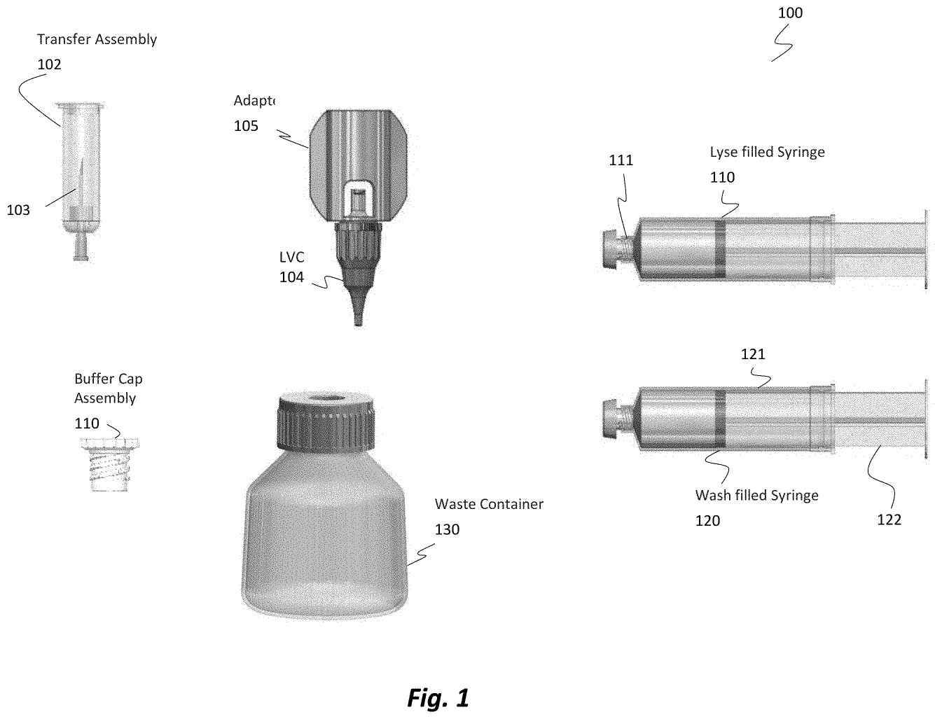

FIG. 1 shows components of an exemplary system or kit for Blood Processing System (BPS) and for sample preparation. Specifically, the exemplary BPS of FIG. 1 includes transfer assembly 102, buffer cap assembly 110, Large Volume Concentrator (LVC) 104 and adaptor 105 (shown coupled, but they may be separate and separatable elements), lyse filled syringe 110, wash filled syringe 120 and waste container 130. The system of FIG. 1 may be used as a kit to implement a blood processing unit according to the disclosed embodiments.

Transfer assembly 102 may be used to transfer biological specimen (e.g., blood) extracted from a patient to the processing system disclosed further below. An exemplary transfer assembly 102 comprises a tubular section having a hollow needle at the base thereof. The needle may be integrated into transfer assembly 102. In another embodiment, the hollow needle portion 103 is assembled onto the cylindrical portion to form transfer assembly 102. In one implementation, the transfer assembly couples to a blood draw tube (not shown) which contains a patient's blood sample. When assembled with the blood draw tube, needle 103 punctures the blood draw tube's cap (not shown) to allow blood specimen to enter transfer assembly tube 102. It should be noted that the illustrated transfer assembly is exemplary and non limiting. Other sample transfer mechanisms may be implemented without departing from the disclosed principles.

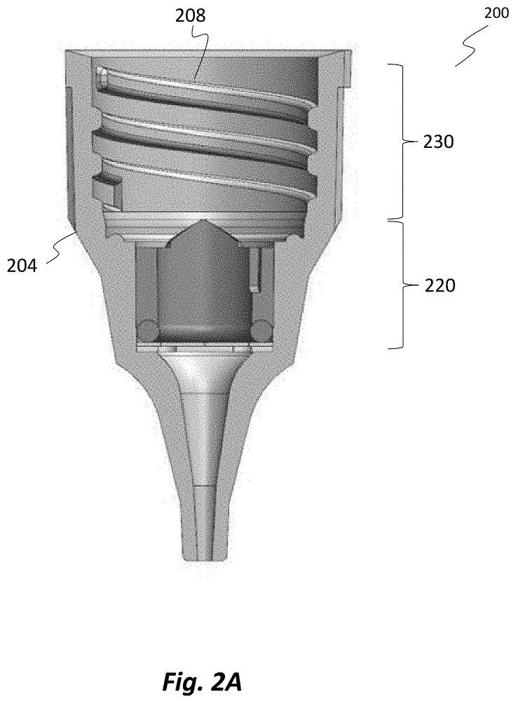

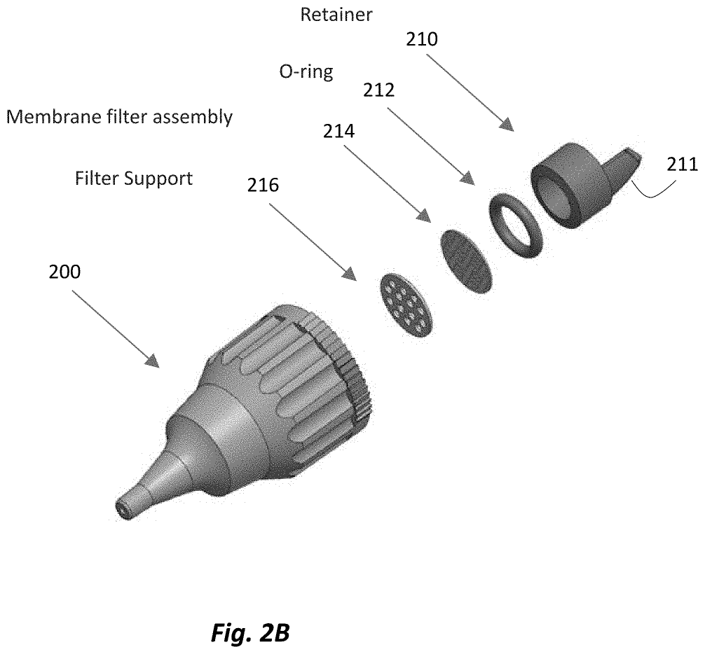

Large Volume Concentrator (LVC) 104 and Adapter 105 are shown as part of system 100 in the assembled representation. The LVC 104, which is discussed in greater detail at FIG. 2, may be used to communicate sample to waste container 130. Lyse field syringe 110 may comprise a cylindrical chamber for receiving lying reagent(s) and a piston to move along a longitudinal axis of the syringe (or syringe cylinder) in order to communicate fluid (e.g., sample and/or reagent) through syringe opening 111. Similarly, wash-field syringe 120 comprises a cylindrical chamber 121 for receiving the wash solution and plunger 122 to move along a longitudinal axis of chamber 121 to communicate wash fluid. The exemplary function of each of syringe 111 and 120 is further illustrated below.

FIG. 2A illustrates an exemplary LVC according to one embodiment of the disclosure. Specifically, FIG. 2A schematically illustrate the inside of the LVC 105 of FIG. 1 and FIG. 2B is an exploded illustration of the LVC of FIG. 2A. With reference to FIGS. 2A and 2B, LVC 200 includes LVC tube housing 204 and threaded portion 208. An exemplary LVC tube housing 204 may receive retainer 210, O-ring 212, membrane filter assembly 214 and filter support 216. Threaded portion 208 may be used to receive an adapter. In some embodiments, threaded portion 208 may be used to couple LVC 200 directly to other components of the system.

Filter support 216 may comprise any suitable material, including inert material, to support membrane filter assembly 214. Membrane filter assembly 214 may be formed of any suitable material with holes, opening, aperture or perforation formed therein. The membrane filter size may be selected to retain pathogenic particles and component while allowing other fluid and material to pass through. O-ring 212 may be placed over membrane filter assembly 214 and filter support 216. Finally, retainer 210 may be inserted over O-ring 212 to keep the entire assembly within LVC 200. Retainer 210 may optionally comprise a notch portion 211. Notch 211 may define a sharp protrusion which extends out of a lateral plane of retainer 210 and extends towards inlet 230 (which may be threaded 208) of the LVC 200. In some embodiment, notch portion 211 may be configured to puncture a surface received at inlet 230 and threaded (or positioned) adjacent to lower portion 220 of LVC 200. As show in FIG. 2A, this entire assembly shown in FIG. 2A may be received and housed at the lower portion 220 of LVC 200.

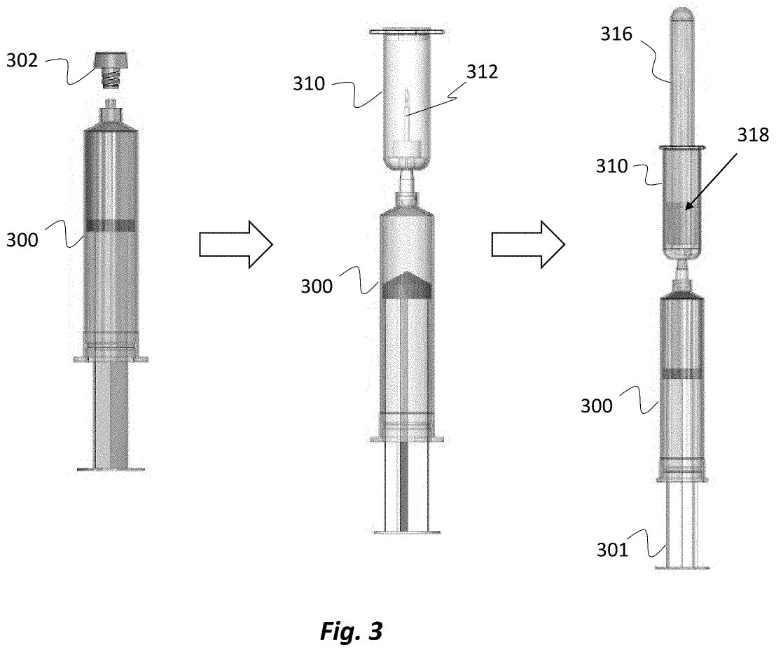

FIG. 3 schematically illustrates the sample extraction steps according to one embodiment of the disclosure. Specifically, FIG. 3 shows the transfer of a patient sample to a lysing syringe using the blood processing system of FIG. 1. At step 1, the twist-off syringe cap 302 is removed from syringe 300 as illustrated. Syringe 300 may contain lysing agent to lyse blood cells. Syringe 300 may also contain buffers or other desired reagents. The lysing agent may comprise conventional lysing agent(s) or may be specifically designed for the desired outcome. Once cap 302 is removed, transfer assembly 310 is coupled to syringe 300. The coupling may be accomplished by pressing the transfer assembly 310 onto the opening of syringe 300. Alternatively, transfer assembly 310 may be coupled to syringe 300 though a threaded portion on the transfer assembly (not shown). Next, a tube containing patient's specimen (e.g., blood) is inserted into transfer assembly 310. The hollow needle 312 of transfer assembly 310 punctures cap 318 of blood draw tube 316, and by withdrawing plunger 301, the sample may be transferred into the barrel of syringe 300. Blood draw tube 316 may be a conventional blood draw tube having a tubular body 316 and a cap 318. One or more reagents and/or buffers (not shown) may be pre-loaded onto the blood draw tube 316 to preserve the integrity of the sample.

Once the sample blood is received at lysing syringe 300, it may be pre-processed by allowing the lysing reagent(s) to react with the sample constituents. In certain embodiments, lysing agents may be used to lyse non-nucleated red blood cells and to preserve white blood cells. This allows white blood cell counts and quantitative determination of hemoglobin of blood samples in clinical settings. Additional processing may be required to complete the lysing step.

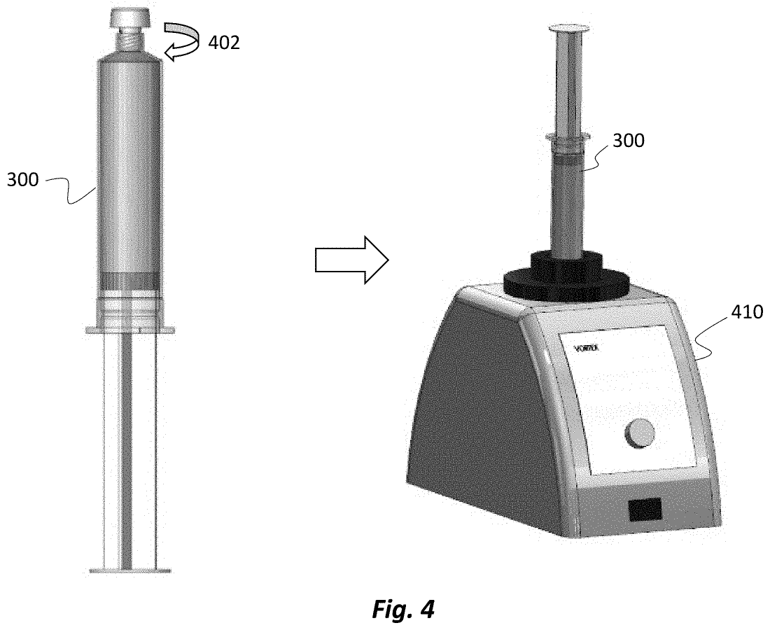

FIG. 4 illustrates an exemplary embodiment for implementing the lysing step according to one embodiment of the disclosure. First, a twist-on cap is placed over the syringe which contains the patient's blood sample. This is illustrated by application of twist-on cap 402 over lysing syringe 300. Next, the capped lysing syringe is placed into agitator 410 for a desired period. Agitator 410 may comprise a vortex, a centrifuge or a sonication device. In an exemplary embodiment where a vortex is used, the vortex device may be activated for a period of, for example, 15, 30, 45, 60, 120, 180 seconds or longer. an exemplary implementation, the syringe and its content (i.e., blood) is vortexed for about 45 seconds at a medium setting. In addition to activating lysing, the vortex agitation may also help disaggregate platelet clumps in the sample. Other means for activating or initiating the lysis reaction within syringe 300 may be used without departing from the disclosed principles. Additional time may be allotted to effectuate the lysing step. Once the disaggregation process is completed, the pathogen components and the non-pathogen components may be separated. In an exemplary embodiment, physical separation via filtration may be used to separate the desired pathogenic components from the blood sample. While FIG. 4 shows a vortex, other agitation means may be used. For example, sonication or centrifugal force maybe exerted to the content of tube 300 to prompt reaction.

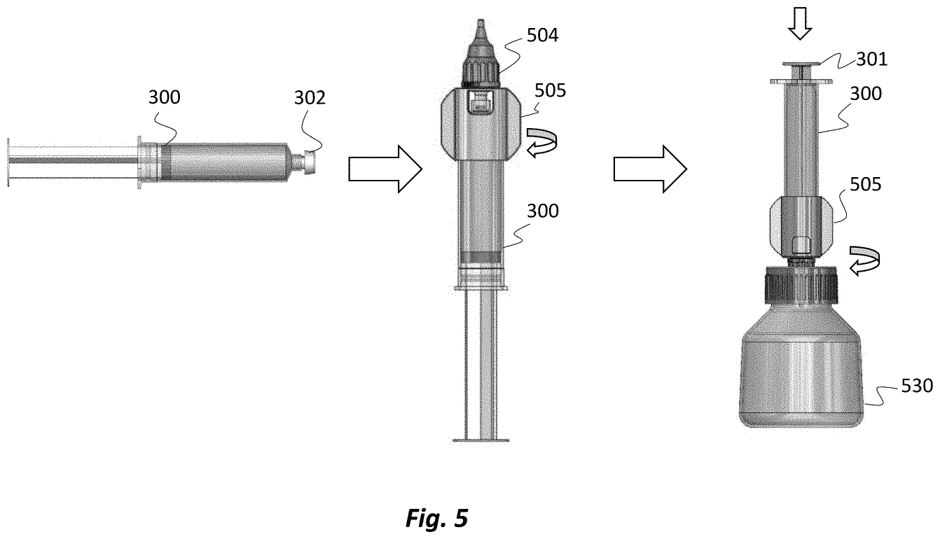

FIG. 5 illustrates the exemplary steps for separating the pathogenic components of interest from the sample according to one embodiment of the disclosure. As illustrated in FIG. 5, lysing syringe 300 containing the processed (e.g., vortexed) sample is allowed to rest for a period to extend lysing. Next, cap 302 is removed from syringe 300 and LVC 504 and adapter 505 assembly are coupled to the lysing syringe 300. The LVC and adapter assembly be coupled to syringe 300 through a twisting mechanism as shown. Next, the adapter-assembled syringe 300 may be coupled to waste container 530 as illustrated (e.g., through a threaded mechanism). Adapter 505 may be used to implement twisting of syringe 300 to waste container 530. Finally, plunger 301 may be forced to eject the syringe content into waste container 530. As the content is forced out of syringe 300 and into waste container 530, the lysed blood cells (not shown) and other particles contained therein (e.g., pathogens) are captured on the filter surface (see membrane filter assembly 214, FIG. 2) of LVC 504.

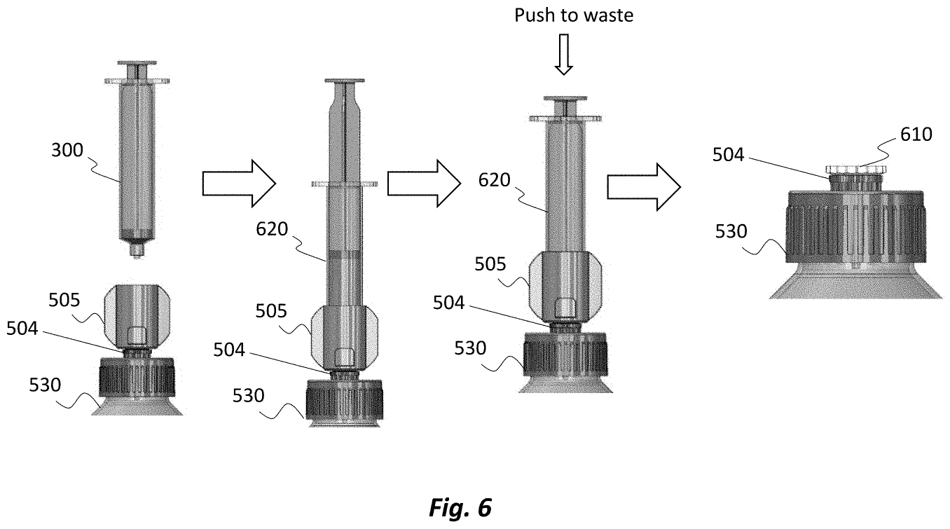

FIG. 6 illustrates the syringe removal and membrane filter wash process according to one embodiment of the disclosure. First, syringe 300, which is now substantially empty of fluid is removed from waste contained 505. Next, wash syringe 300 is coupled to waste container 620. Wash syringe 620 may contain washing fluid to wash the membrane filter assembly inside LVC 106. The washing fluid is moved through adapter 505 and LVC 504 in order to wash the membrane filter (nots shown) inside LVC 504. The washing fluid may comprise one or more buffer solutions to wash unwanted particles off the membrane filter. Once the content of the washing syringe 620 are emptied, the syringe may be decoupled from waste container 530. Finally, buffer cap 610 may be placed over LVC 504. The membrane filter (not shown) of LVC 106 may now have captured particles of interest thereon. The membrane may be removed (not shown) at subsequent steps for further particle analysis.

The following non-limiting examples illustrate various embodiments and applications of the disclosed principles. These examples are illustrative of the disclosed principles and are not exhaustive nor limiting.

Example 1 is directed to a system to detect one or more blood-borne pathogens, the system comprising: a transfer assembly having a tube and a hallow needle, the hallow needle centrally located within the transfer assembly tube and configured to communicate a sample material therethrough; a lysing syringe to couple to the transfer assembly, the lysing syringe comprising one or more lysing reagent and a plunger activatable to receive the sample material through the transfer assembly; and a large volume concentrator (LVC) to sealingly couple to the lysing syringe and to separate at least one pathogen from the sample material, the LVC further comprising: a filter support, a membrane, a retainer and a threaded portion; wherein the retainer is configured to secure the membrane against the filter support.

Example 2 is directed to the system of Example 1, further comprising an adapter to couple to the LVC, the adapter configured to allow a twisting motion along a longitudinal axis thereof.

Example 3 is directed to the system of Example 1, further comprising an agitator to receive the lysing syringe containing the sample material.

Example 4 is directed to the system of Example 3, wherein the agitation apparatus is selected from the group consisting of a vortex, a centrifuge or a sonication device.

Example 5 is directed to the system of Example 1, wherein the LVC further comprises a notch with a sharp protrusion which extends longitudinally towards an inlet of the LVC.

Example 6 is directed to the system of Example 1, wherein the lysing syringe sealingly couples to the transfer assembly.

Example 7 is directed to the system of Example 1, wherein the membrane is sized to collect one or more bloodborne pathogens.

Example 8 is directed to the system of Example 1, wherein the transfer assembly is activatable to receive the sample material from a blood draw tube through the transfer assembly.

Example 9 is directed to a method to detect presence of one or more bloodborne pathogens, the method comprising: communicating a sample material to a lysing syringe through a transfer assembly, the transfer assembly having a tube and a hallow needle, the hallow needle centrally located within the transfer assembly tube; lysing one or more sample components at the lysing syringe by contacting the sample material with one or more lysing reagent to form a lysed sample; and filtering the lysed sample through a membrane of a large volume concentrator (LVC) to isolate at least one pathogen from the lysed sample and to thereby form the lysed sample, the LVC further comprising: a filter support, the membrane, a retainer and a threaded portion; and washing the membrane of the LVC with a wash fluid; wherein lysing one or more sample components further comprises agitating the sample material and the one or more lysing agents.

Example 10 is directed to the method of Example 9, further comprising mechanically coupling an adapter to the LVC, the adapter configured to allow a twisting motion along a longitudinal axis thereof to thereby couple the LVC to the waste container.

Example 11 is directed to the method of Example 9, wherein agitating the sample material further comprises agitating the sample material and the lysing reagent to promote lysing of at least one component of the sample material.

Example 12 is directed to the method of Example 11, wherein agitating the sample material further comprises one of vortexing, centrifuging or sonicating the sample material for a duration.

Example 13 is directed to the method of Example 9, wherein the LVC further comprises a notch with a sharp protrusion which extends longitudinally towards an inlet of the LVC.

Example 14 is directed to the method of Example 9, further comprising coupling the lysing syringe to the transfer assembly.

Example 15 is directed to the method of Example 9, further comprising selecting a membrane configured to collect at least one bloodborne pathogen from the sample material.

While the principles of the disclosure have been illustrated in relation to the exemplary embodiments shown herein, the principles of the disclosure are not limited thereto and include any modification, variation or permutation thereof.

What is claimed is:

1. A system to detect one or more blood-borne pathogens, the system comprising:
a transfer assembly having a tube and a hallow needle, the hallow needle centrally located within the transfer assembly tube and configured to communicate a sample material therethrough;
a lysing syringe to couple to the transfer assembly, the lysing syringe comprising one or more lysing reagent and a plunger activatable to receive the sample material through the transfer assembly; and
a large volume concentrator (LVC) to sealingly couple to the lysing syringe and to separate at least one pathogen from the sample material, the LVC further comprising: a filter support, a membrane, a retainer and a threaded portion;
wherein the retainer is configured to secure the membrane against the filter support.

2. The system of claim 1, further comprising an adapter to couple to the LVC, the adapter configured to allow a twisting motion along a longitudinal axis thereof.

3. The system of claim 1, further comprising an agitator to receive the lysing syringe containing the sample material.

4. The system of claim 3, wherein the agitation apparatus is selected from the group consisting of a vortex, a centrifuge or a sonication device.

5. The system of claim 1, wherein the LVC further comprises a notch with a sharp protrusion which extends longitudinally towards an inlet of the LVC.

6. The system of claim 1, wherein the lysing syringe sealingly couples to the transfer assembly.

7. The system of claim 1, wherein the membrane is sized to collect one or more bloodborne pathogens.

8. The system of claim 1, wherein the transfer assembly is activatable to receive the sample material from a blood draw tube through the transfer assembly.

9. A method to detect presence of one or more bloodborne pathogens, the method comprising:
communicating a sample material to a lysing syringe through a transfer assembly, the transfer assembly having a tube and a hallow needle, the hallow needle centrally located within the transfer assembly tube;
lysing one or more sample components at the lysing syringe by contacting the sample material with one or more lysing reagent to form a lysed sample; and
filtering the lysed sample through a membrane of a large volume concentrator (LVC) to isolate at least one pathogen from the lysed sample and to thereby form the lysed sample, the LVC further comprising: a filter support, the membrane, a retainer and a threaded portion; and
washing the membrane of the LVC with a wash fluid;
wherein lysing one or more sample components further comprises agitating the sample material and the one or more lysing agents.

10. The method of claim 9, further comprising mechanically coupling an adapter to the LVC, the adapter configured to allow a twisting motion along a longitudinal axis thereof to thereby couple the LVC to the waste container.

11. The method of claim 9, wherein agitating the sample material further comprises agitating the sample material and the lysing reagent to promote lysing of at least one component of the sample material.

12. The method of claim 11, wherein agitating the sample material further comprises one of vortexing, centrifuging or sonicating the sample material for a duration.

13. The method of claim 9, wherein the LVC further comprises a notch with a sharp protrusion which extends longitudinally towards an inlet of the LVC.

14. The method of claim 9, further comprising coupling the lysing syringe to the transfer assembly.

15. The method of claim 9, further comprising selecting a membrane configured to collect at least one bloodborne pathogen from the sample material.
